(19) United States
(12) Patent Application Publication
(10) Pub. No.: US 2006/0106391 A1
(43) Pub. Date: May 18, 2006

(54) Wire systems for fixing bones

(76) Inventor: Randall J. Huebner, Beaverton, OR (US)

Correspondence Address: KOLISCH HARTWELL, P.C., 200 PACIFIC BUILDING, 520 SW YAMHILL STREET, PORTLAND, OR, 97204, US

(21) Appl. No.: 11/273,811

(22) Filed: Nov. 14, 2005

Related U.S. Application Data
(60) Provisional application No. 60/627,297, filed on Nov. 12, 2004.

(51) Int. Cl. A61B 17/56

(52) U.S. Cl. 606/74

(57) ABSTRACT

Systems, including methods, apparatus, and kits, for fixing bones with wires having longitudinally arrayed segments of different flexibility.

### CROSS-REFERENCE TO PRIORITY APPLICATION

This application is based upon and claims the benefit under 35 U.S.C. § 119(e) of the following U.S. provisional patent application, which is incorporated herein by reference in its entirety for all purposes: Ser. No. 60/627,297, filed Nov. 12, 2004.

### CROSS-REFERENCES TO OTHER MATERIALS

This application incorporates by reference in their entirety for all purposes the following U.S. Pat. No. 5,697,934, issued Dec. 16, 1997; and U.S. Pat. No. 6,017,347, issued Jan. 25, 2000.

### INTRODUCTION

The human skeleton is composed of 206 individual bones that perform a variety of important functions, including support, movement, protection, storage of minerals, and formation of blood cells. To ensure that the skeleton retains its ability to perform these important functions, and to reduce pain and disfigurement, bones that become damaged should be repaired promptly and properly.

Typically, a fractured or cut bone is treated using a fixation device, which reinforces the bone and keeps it aligned during healing. External fixation may be achieved using casts and/or fixators, among others, which are minimally invasive and allow reduction and fixation of simple fractures from outside the body. Internal fixation may be achieved using bone plates, bone screws, and/or wires, among others. Bone plates mount directly to bone adjacent fractures, for example, using bone screws or wires. Bone screws also may be placed into bone without the use of bone plates, so that the screws span a fracture and fix fractured bones. Wires generally wrap around fractured bones, binding together fragments of each bone and holding them in place while the bone heals.

Wires used to bind fractured bones should be easy to manipulate before and during placement around bone and should be sufficiently flexible to bind bones properly after this placement. However, wires with a suitable flexibility for bone fixation may be too flexible for easy manipulation. For example, these wires may be difficult to feed through soft-tissue incisions so that the wires wrap around bone. Accordingly, these wires tend to develop kinks and bends when handled during surgery, making them difficult to wrap around bones and/or to thread through apertures in wire clamps or bone plates.

### SUMMARY

The present teachings provide systems, including methods, apparatus, and kits, for fixing bones with wires having longitudinally arrayed segments of different flexibility.

### BRIEF DESCRIPTION OF THE DRAWINGS

FIG. 1 is a view of a fractured bone fixed with an exemplary wire system, in accordance with aspects of the present teachings.

FIG. 2 is a view of the wire system of FIG. 1, including a wire of varying flexibility and a locking mechanism received on the wire, prior to placement of the wire around the fractured bone and prior to actuation of the locking mechanism.

FIG. 3 is a fragmentary view of an appendage having a fractured bone and an access opening formed in soft tissue overlying the fractured bone.

FIGS. 4-10 are sectional views of the appendage of FIG. 3, taken generally along line 4-4 of FIG. 3, with the appendage in various configurations produced by performing an exemplary method of fixing the fractured bone with the wire system of FIG. 2, in accordance with aspects of the present teachings.

### DETAILED DESCRIPTION

The present teachings provide systems, including methods, apparatus, and kits, for fixing bones with wires having a varying flexibility. The wire systems may include a wire having at least two longitudinal regions or segments of different flexibility. The different flexibility may result from a difference in the intrinsic flexibility of a material forming each segment and/or from a difference in shape and/or size of each segment. The segments may be included in a unitary wire, such as by selectively treating (e.g., selectively heating and/or cooling) a longitudinal portion of the unitary wire, or may be formed as separate components that are secured to one another. In some examples, the wire may include a relatively stiffer, less bendable guide segment and a relatively more flexible/bendable fixation segment connected to the guide segment. In some embodiments, each of the guide and fixation segments may have a length that is greater than the circumference of a bone to be fixed.

Methods of using the wire systems of the present teachings may include guiding the more flexible fixation segment into position around a fractured bone through selective engagement and manipulation of the guide segment. For example, the stiffer guide segment may have a length and stiffness sufficient to serve as a non-kinking handle for manipulation of the wire during surgery. The stiffer guide segment thus may be pushed and/or pulled so that the guide segment leads a proximal region of the more flexible fixation segment through a soft-tissue opening, around a fractured bone, and, optionally, at least partially out of the opening. The fixation segment then may be secured in position on the bone to fix the fractured bone. In some examples, the wire may be cut to remove the guide segment at least substantially from the remainder of the wire after the guide segment has performed its guiding function. The wire systems of the present teachings thus may provide easier manipulation of wires during placement around bone without sacrificing wire flexibility during fixation.

Further aspects of the invention are described in the following sections, including, among others, (I) overview of wire systems, (II) wires, (III) methods of making wires of varying flexibility, and (IV) methods of using wires of varying flexibility.

I. Overview of Wire Systems

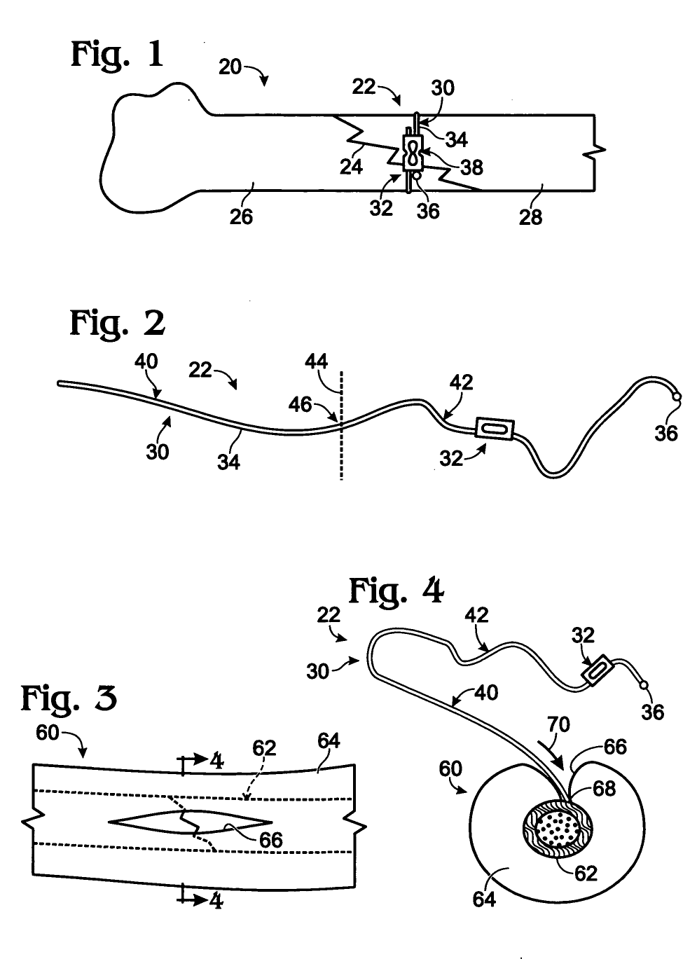

FIG. 1 shows a fractured bone 20 fixed with an exemplary wire system 22. Bone 20 may include a fracture 24 that divides the bone 20 into two or more bone pieces 26, 28. The wire system may wrap at least substantially or completely around the bone, one or more times, to bind bone pieces 26, 28 together and thus fix the bone. In some examples, the wire system also may include an implant, such as a prosthesis, a bone plate, a rod, and/or the like, to be secured to the bone. Alternatively, or in addition, the wire system may wrap around more than two bone pieces and/or two or more bones.

Wire system 22 may include a wire 30 and locking or clamp mechanism 32 (a retainer) to hold end regions of the wire in position on the bone. The wire 30 may have an elongate body 34 and a stop structure 36 disposed adjacent and/or forming an end (or both ends) of the wire. The elongate body may have sufficient length to wrap at least substantially around the bone, at least once, twice, or more times. The stop structure may be configured to engage the locking mechanism, for example, to restrict wire from sliding out of the locking mechanism. Alternatively, or in addition, the stop structure may be configured to be engaged by hand or with a tool, to facilitate manipulation of the wire. The locking mechanism may be actuable selectively, for example, by placing a crimp, shown at 38, in a locking sleeve of the mechanism, to engage one or both end regions of the wire, so that one or both end regions are clamped in position by the locking mechanism.

FIG. 2 shows wire system 22 prior to placement of the wire 30 around the fractured bone and prior to actuation of the locking mechanism. Wire 30 may include two or more longitudinally arrayed segments of different flexibility. A relatively stiffer, less bendable guide segment 40 may be disposed toward one end of the wire, and a relatively more flexible and bendable fixation segment 42 may be disposed toward the other end of the wire. An approximate boundary between these segments is indicated here with a dashed line 44. The guide segment 40 may be removed from the remainder of the wire during and/or after placement of the wire around the bone, and thus is not present in the shortened form of wire body 34 shown in FIG. 1. Accordingly, stop structure 36 may be disposed adjacent fixation segment 42 and spaced from guide segment 40.

Wire 30 also may include a junction region 46 disposed between the segments of different flexibility. The junction region may define a transition zone of intermediate and/or varying flexibility and/or a site at which the guide and fixation segments are joined to one another, either unitarily or after their formation as separate components.

The locking mechanism 32 may be coupled to the wire before, during, or after the wire is placed in position around the bone. In the present illustration, the locking mechanism includes a pair of adjacent sleeves 46 defining channels in which the wire may be slidably received. The sleeves may be deformed, as shown in FIG. 1, at any suitable time to actuate the locking mechanism and thus fix the wire to the locking mechanism. The locking mechanism shown here is exemplary. Any suitable locking or clamp device, or wire retainer, may be used and/or the wire may be twisted or wrapped around itself and/or an implant so that it is secured.

II. Wires

The wires of the present teachings may have any suitable structure, segments, and composition, and as described below.

A. Wire Structure

The wires described herein may have any suitable size and/or shape consistent with their use in orthopedic applications. Moreover, size and/or shape may vary, from wire to wire, and from position to position on a given wire, depending on the application and/or the patient, among others. For example, the wire may be relatively thinner and/or shorter for smaller bones and/or patients, or relatively thicker and/or longer for larger bones and/or patients.

A wire may have any suitable cross-sectional shape and characteristic width(s). For example, the cross-sectional shape of the wire may be circular, oval, elliptical, square, rectangular, triangular, trapezoidal, rhomboidal, and/or irregular, among others. Moreover, the cross-sectional shape may be constant and/or variable along the length of the wire. Similarly, the characteristic width(s) or diameter of the wire may be constant and/or variable. In some examples, the wire has a circular cross-section, and a diameter that is significantly smaller than the diameter of the bone. In some examples, the wire may have a diameter of about 0.1 mm to 5 mm, or about 0.5 mm to 3 mm, among others. Exemplary illustrative diameters include, but are not limited to, 0.7 mm, 0.9 mm, 1.1 mm, 1.6 mm, 2.0 mm, 2.4 mm, and 2.8 mm, among others.

A wire also may have any suitable length. Typically, the length is at least about several times larger than the diameter of the bone and at least about as great as the circumference of the bone, to allow the wire to be wrapped around the bone or a portion thereof, one or more times. The wire may be of a relatively shorter or longer length, depending on the application (e.g., small bone or large bone), fracture or other indication (e.g., near a thin or wide section of bone), and so on. In some examples, the wire may have a length of about 10 to 150 cm, or about 50 to 100 cm, among others. In exemplary illustrative embodiments, the length may be about 85 cm. The length may differ before and after fixation, since an extra length of the wire may be used to facilitate handling during fixation, and then removed following fixation. The length of the wire may be at least about 10, 20, 50, or 100 times its diameter, among others.

A wire may be solid or hollow. If hollow, the wire generally may have one or more cavities, positioned along part or all of its length. These cavities may have any suitable cross-sectional shape, width, and/or length. For example, the cavities may be circular, ovoid, square, rectangular, triangular, trapezoidal, rhomboidal, cruciform, and/or irregular, among others. The wire may be uniformly or variably hollow throughout its length, partly hollow and partly solid, and/or alternately hollow and solid. In some examples, the wire may be cannulated, having an axial bore that extends along part or all of its length. The cross-sectional shape of the hollow portion(s) may be the same as, similar to, or different from the cross-sectional shape of the outer portion of a hollow wire.

The body of the wire may have any suitable configuration. For example, the body may be formed unitarily, such as in a monofilament wire. Alternatively, the body may be formed of two or more components, arrayed longitudinally (see sub-section B below) or laterally (e.g., a multi-stranded wire).

The wire may include manipulation features attached to, or included in, the body, and characterized at least in part by a change in size and/or shape (and/or other properties). These manipulation features may be formed as an enlarged region of the wire, such as a bead, loop, or other structure, and may function as stop elements. For example, a bead or loop attached to an end or other portion of the wire body may hold the wire in a wire clamp or other retainer by preventing the wire end from sliding through an aperture of the clamp or retainer. Alternatively, or in addition, these features may facilitate engagement of the wire, such as by hand or with a tool, during installation of the wire.

A manipulation feature may have the same or a different composition than the elongate body of the wire. Accordingly, the manipulation feature may be formed by altering the shape of an end of the wire, such as by melting and/or forming the end or by bending/twisting the wire back on itself to form a loop, ball, or kinked/twisted bundle, among others. Alternatively, the manipulation feature may be formed separately and then secured to the wire body.

A manipulation feature may have any suitable size. For example, the feature may be large enough to prevent the wire from slipping through apertures in wire clamp(s) or retainers that may be used to secure the wire. Alternatively, or in addition, the feature may be large enough to be engaged selectively with a tool.

A manipulation feature may have any suitable shape and property. Exemplary shapes may include spherical, square, loop-like, or rectangular, among others. The feature may be hollow or solid, and flexible or inflexible.

When the feature is a loop, the loop may be at either or both ends of the wire. If the wire is to be threaded through apertures in wire clamps, bone plates, or other fracture fixation apparatus, the loop at the threading end may be flexible, such that the loop may be flattened to fit through the aperture(s).

B. Wire Segments

The wires of the present teachings may include at least two longitudinal segments of different flexibility. In particular, one of the segments may be relatively more flexible (and relatively less stiff) than another of the segments. Alternatively, or in addition, one of the segments may have a greater intrinsic flexibility than another segment of the wire, based on the intrinsic flexibility of the material forming each segment. Flexibility and/or intrinsic flexibility between segments of a wire (and/or materials thereof) may differ by any suitable amount, for example, by at least about 10%, 25%, 50% 75%, 2-fold, or 4-fold, among others.

Flexible (or flexibility), as used herein, refers generally to an ability to be bent, twisted, or turned, typically repeatedly, and often without undergoing functionally significant change or modification. Thus, a relatively more flexible wire segment (or material) can be bent, twisted, or turned more easily and/or to a greater extent than a relatively less flexible wire segment (or material). Flexibility can be described or quantified using any suitable function(s) or parameter(s). A description of a segment's flexibility may involve a combination of one or more intrinsic, material-dependent properties of the segment and one or more extrinsic, material-independent properties of the segment. Exemplary intrinsic properties may include measures, such as Young's modulus (describing the relationship between stress and strain), of how the material(s) forming the segment responds to applied mechanical forces. Exemplary extrinsic properties may include geometrical properties of the segment, such as the area moment of inertia (pertinent to bending) or polar moment of inertia (pertinent to twisting), which provide measures of the distribution or arrangement of material in the segment. Generally, materials with relatively smaller or lower Young's moduli or moments of inertia are more flexible (or less stiff) than materials with relatively larger or higher Young's moduli or moments of inertia. Exemplary Young's moduli may include about 20×10¹⁰ N/m² for some forms of steel, about 7×10¹⁰ N/m² for some forms of aluminum, and about 0.0001×10¹⁰ N/m² for some forms of rubber, among others. Exemplary area moments of inertia may include πr⁴/4 for a solid cylinder of radius r and π(a⁴−b⁴)/4 for a hollow cylinder of outer radius a and inner radius b, among others. (These latter equations illustrate, in the context of cylinders, the more general principle that flexibility decreases as size or extent increases.) For a given shape and size, materials associated with greater flexibilities (e.g., materials with lower Young's moduli) can be referred to as having greater intrinsic flexibilities, and materials associated with lesser flexibilities (e.g., materials with higher Young's moduli) can be referred to as having lesser intrinsic flexibilities.

The segments of a wire may have any suitable size and shape. The segments may have at least substantially the same diameter or may have different diameters. For example, a relatively more flexible segment in the wire may have a smaller diameter than a relatively less flexible segment. In some examples, the diameters of two or more segments of a wire may differ by less than about 20% or 10%, among others. Two or more segments of a wire may have lengths that differ by less than about two-fold and/or may be of about the same length. In some embodiments, two or more segments of a wire may have lengths that differ by greater than about two-fold or greater than about five-fold, such as a relatively more flexible segment flanked by one or more short leader segments that are relatively less flexible. Two or more segments of a wire may have distinct (e.g., circular and elliptical, among others) or substantially the same cross sectional geometries.

The segments of a wire may have any suitable appearance and disposition. The appearance of the segments may be about the same or different. For example, one or more of the segments may be marked to facilitate identification of relatively more and/or less flexible regions of the wire. Exemplary markings on or adjacent one or more segments include a marked surface (such as a visible coating, a band(s), a dot(s), an alphanumeric character(s), a colored indicium or region, etc.), a surface structure (such as a ridge, a groove, a depression, a roughened surface, etc.), a knot, and/or a manipulation feature (such as a bead, loop, etc.), among others. The segments may be disposed generally at opposing end regions of the body of the wire, and may be separated by a junction region of any suitable size (or no substantial junction region). The junction region may have a flexibility distinct from the flexibilities of adjacent segments, for example, a flexibility intermediate between that of the adjacent segments. In some examples, a relatively more (and/or less) flexible segment may be flanked on both ends by relatively less (and/or more) flexible segments.

C. Materials

The wire described herein may be formed of any suitable material(s), particularly one or more biocompatible material(s). Exemplary biocompatible materials that may be included in, and/or at least substantially may form, the wire include (1) metals/metal alloys (for example, titanium or titanium alloys; alloys with cobalt, chromium, and/or molybdenum (such as cobalt-chrome); stainless steel; etc.); (2) plastics (such as ultra-high molecular weight polyethylene, polymethylmethacrylate (PMMA), polytetrafluoroethylene (PTFE), and/or PMMA/polyhydroxyethylmethacrylate (PHEMA)); and/or (3) bioabsorbable polymers (such as polymers of α-hydroxy carboxylic acids (e.g., polylactic acid (such as PLLA, PDLLA, and/or PDLA), polyglycolic acid, lactide/glycolide copolymers, etc.), polydioxanones, polycaprolactones, polytrimethylene carbonate, polyethylene oxide, poly-β-hydroxybutyrate, poly-β-hydroxypropionate, poly-δ-valerolactone, poly(hydroxyalkanoate)s of the PHB-PHV class, other bioresorbable polyesters, natural polymers (such as collagen or other polypeptides, polysaccharides (e.g., starch, cellulose, and/or chitosan), any copolymers thereof, etc.); and/or the like.

In some examples, the wire may include or be formed at least substantially of stainless steel. Stainless steel, as used herein, is any iron alloy that is corrosion resistant. In addition to iron, stainless steel may include carbon and/or chromium, among others. In some embodiments, other elements may be included, for example to alter the properties of the steel, such as copper; nitrogen; nickel for increased corrosion resistance, ductility, and workability; molybdenum for increased resistance to corrosion and pitting, and increased high-temperature strength; columbium and titanium for stabilization; and/or sulfur and selenium for improved machinability, among others. Exemplary illustrative wires may be formed of stainless steel that is Type 316, LVM medical grade.

In some examples, the wire may include or be formed at least substantially of a superelastic and/or shape memory material, such as a nickel titanium alloy. An exemplary illustrative material with these properties is Nitinol™ alloy, among others. Nickel titanium alloys may contain about 50 atomic percent each of nickel and titanium (about 55 percent nickel by weight). However, subtle adjustments in the ratio of the two elements may affect the properties of the nickel titanium alloy, especially its transformation temperatures. These transformation temperatures correspond to the temperatures at which the crystal structure of the alloy changes from austenite to martensite or vice versa. Austenite refers to the stronger, higher-temperature phase of a nickel titanium alloy, while martensite denotes the more plastic, lower-temperature phase. The shape memory property allows objects made of nickel titanium alloy to regain their former shapes after physical deformation at lower temperatures.

The wire may include a coating extending along some or all of it length. The coating may be formed of a different material than the underlying core and may be configured to mark the wire (or a portion thereof), reduce friction, alter the bioabsorbability of the wire (or a portion thereof), and/or reduce corrosion, among others. Exemplary coatings may include a bioabsorbable material and/or may a color different distinct from the underlying core.

The segments of the wire each may be formed of any suitable material. The composition of the material may remain substantially the same or may vary along the length of the wire. Composition, as used herein, relates to the atomic composition and molecular formula of the material.

Two or more segments of the wire each may be formed of a material with substantially the same composition. For example, two or more segments of a wire may be formed of substantially the same material but may differ in flexibility due to differences in treatment (such as differences in annealing, cold-working, etc.) of the material that alter its intrinsic flexibility. The material of two or more segments thus may be chemically the same, but may have different crystal structures or crystal microstructures (such as different sizes/shapes/dispositions of crystal grains) within the material in the segments. Alternatively, or in addition, segments formed of the same material may differ in flexibility due to differences in dimension and/or shape of the material (such as segments that differ in diameter or cross-sectional shape).

In some embodiments, the composition of the wire may differ between segments. Relatively more and less flexible segments of the wire thus may be made of different materials with different intrinsic flexibilities, such as, for example, stainless steel and nickel titanium alloy, among others. A junction region between these segments, if present, may be formed by a mixture of these different materials (such as with a unitary wire body), by a distinct material, by a joint created by the unmixed materials of the segments, and/or the like.

III. Methods of Making Wires of Varying Flexibility

The wires of varying flexibility described herein may be manufactured in any suitable manner. The following description, outlining general methods of manufacture, is included for illustration and is not intended to limit or define the entire scope of the present teachings. This description focuses on the manufacture of multi-segment wires from a single composition (unitary wires), or from separately formed segments having different compositions (non-unitary wires).

A. Unitary Wires

A wire with varying flexibility may be formed by differential processing of a unitary wire along its length. Exemplary differential processing may include selectively treating a longitudinal portion of the wire, so that this portion is selectively hardened (rendered less flexible) and/or softened (rendered more flexible). Hardening may be performed, for example, by cold-working (such as by applying pressure to the wire with a die), suitable heating (such as in a bath or oven, with a flame, using a laser, and/or with an electron beam, among others), suitable cooling (such as in a bath or refrigerator, among others), and/or the like. Softening may be performed, for example, by annealing, among others. Annealing, as used herein, involved heating a material above its recrystallization temperature (and generally below its melting temperature), and then cooling the material, generally slowly, so that the microstructure of the material changes. Accordingly, the duration, rate, and/or temperature of heating and/or cooling, and the particular material being treated, may determine whether the heating and/or cooling results in hardening or softening of the material. In exemplary embodiments, a longitudinal region(s) of a metal wire may be hardened selectively by cold-working this region(s), to form a relatively less flexible segment(s). Alternatively, or in addition, a longitudinal region(s) of the wire may be softened selectively by annealing this region(s), to form a relatively more flexible segment(s).

In some embodiments, the differential processing of the wire may produce segments with different diameters or characteristic widths. For example, the wire may be formed by extrusion through a die of varying diameter, and/or a longitudinal region of the wire may be selectively stretched along its long axis. Alternatively, or in addition, a region of smaller diameter and lower intrinsic flexibility may be formed within the wire by selectively cold-working this region.

B. Non-Unitary Wires

A wire with varying flexibility may be formed by joining two or more wire segments that are formed separately. The segments of the wire may be joined using any suitable method including welding, bonding, riveting, clinching, brazing, and/or thermal joining, among others. Alternatively, or in addition, the segments of the wire may be joined by engaging their ends, such as by twisting the ends together. The wire segments may have different flexibilities due to different compositions, different characteristic widths, and/or differential processing before (and/or after) joining, among others.

In exemplary embodiments, the wire according to the present teachings may also be made from two types of metal alloys, such as stainless steel and nickel titanium alloy. Nickel titanium alloy wire segments may be obtained commercially and/or manufactured from a suitable nickel titanium alloy.

Nickel titanium alloy wire may be useful in fracture fixation when configured to resist development of kinks and bends during handling. Additionally, nickel titanium alloy may be configured as a shape memory alloy, so it may revert to its former shape after a deformation, depending on temperature. For example, a wire/wire segment formed of a nickel titanium alloy may be cooled and stretched before surgery, lengthening the wire. During surgery, the wire may be installed and tightened around bone in its lengthened form. After surgery, the comparatively warmer temperature of the body may cause the nickel titanium alloy wire to revert to its original, shorter length. By fixing the ends of the wire (e.g., by tying, twisting, fixing with wire clamps, or affixing to pins, among other methods of fixation), the wire thus may produce sustained tension in the wire throughout the recovery process, keeping the fractured pieces of bone pulled together tightly and promoting more effective healing.

IV. Methods of Using Wires of Varying Flexibility

The wire systems described herein may be used on any suitable bones or bone portions of the human body or other vertebrate species, for any suitable purpose. Suitable bones may include long bones, among others. Exemplary bones for use with the wire systems described herein may include bones of the arms (radius, ulna, humerus), legs (femur, tibia, fibula, patella), hands, feet, vertebrae, pelvic bones, cranial bones, clavicles, the scapula, and/or the ribs, among others. Exemplary uses of the wire may include fracture fixation, bone stabilization, osteotomy repair, fusion of two or more bones, and/or attachment of implants (such as bone plates, prostheses, rods, bone screws, and/or the like) to a bone(s), among others.

The wire, used to wrap and fixate a fracture in a bone, may be used alone, wrapped around a plate that extends across the fracture, and/or held in place wire clamps and/or pins, among other applications. For example, the wire systems described herein may include a locking sleeve, such as described in U.S. Pat. No. 6,017,347, issued Jan. 25, 2000, which is incorporated herein by reference. Alternatively, or in addition, the wire systems may include tension band wiring pins, such as those described in U.S. Pat. No. 5,697,934, issued Dec. 16, 1997, which is incorporated herein by reference.

Wire of the wire systems may be supplied individually and/or in a kit. The kit may include a plurality of wires of similar or distinct configurations. Among the wires in a kit, distinct wires may be distinguishable (and/or different) based on identifying indicia on the wire, their lengths, diameters, cross-sectional shapes and/or other geometrical characteristics, their physical compositions, the number of segments in each wire, the relative flexibility of each wire, the relative hardness of each wire, and/or the degree of shape memory of each wire, among others. The kit also may include a hole-forming tool (such as a drill, a reamer, drill bit(s), and/or the like), a driver, a wire tensioner, bone plates, wire clamps, prostheses, wire cutters, pliers, and/or instructions for the use of the wires and kit contents, among others.

FIGS. 3-10 illustrate configurations produced before, during, and/or after performing an exemplary method of fixing a fractured bone with an exemplary wire system. The method steps represented by the configurations and/or described below may be performed in any suitable combination, in any suitable order, any suitable number of times.

FIG. 3 shows an appendage 60 having a fractured bone 62 (shown in dashed outline) and soft tissue 64 overlying the fractured bone. An opening 66 may be formed in the soft tissue to provide access to bone 62. The opening may be formed by trauma (e.g., when the bone breaks), surgically (e.g., with a knife), and/or the like. The opening may be disposed asymmetrically relative to the long axis of the bone, so that the bone is not exposed circumferentially. Accordingly, a wire cannot be wrapped around the bone readily with a winding motion.

FIG. 4 shows a sectional view of the appendage of FIG. 3, taken generally along line 4-4 of FIG. 3, with exemplary wire system 22 positioned for placement around the bone. In particular, a distal end region 68 of wire 30, at an end of guide segment 40, may be placed first through tissue opening 66, so that the guide segment leads fixation segment 42 through the opening and around the bone. Clamp device 32 may be disposed on the wire at this stage or may be placed on the wire at any suitable time later in the method, such as after placement of the fixation segment of the wire around the bone. The clamp device may be slidable on the fixation segment at this stage in both axial directions of the wire, may be abutted against stop structure 36 for slidability in only one of the axial directions, or may be fixed to the wire and thus not slidable.

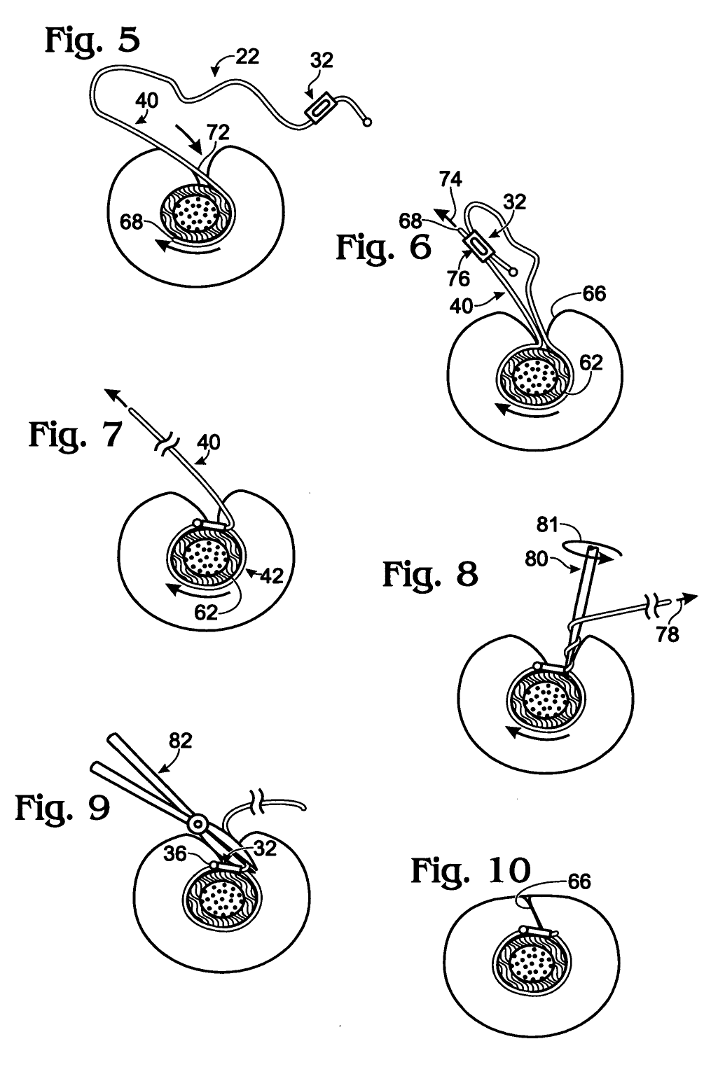

The guide segment of the wire, due to its stiffness, may be engaged manually and/or with a tool, and pushed through the opening and around the bone, in a direction shown at 70. For example, FIG. 5 shows guide segment 40 with its leading end region 68 about half way around the bone. Similarly, FIG. 6 shows the wire after leading end region 68 of guide segment 40 has substantially circumnavigated the bone and passed outward through tissue opening 66. The guide segment thus may be longer than the circumference of bone 62, so that the guide segment can be pushed around the bone by engagement of its proximal region 72 (see FIG. 5). Pushing may be performed until leading end region 68 has traveled at least substantially around the bone and becomes sufficiently exposed near the tissue opening to be engaged and pulled away from the opening, indicated generally at 74 in FIG. 6. At any suitable time, distal end region 68 of the guide segment may be slidably received by clamp device 32, shown at 76. In some examples, the clamp device may be configured to be received laterally on the wire (rather than the wire threaded into the clamp device from an end of the wire) at any suitable along the wire.

FIG. 7 shows fixation segment 42 of the wire wrapped around the bone. The fixation segment may be placed in this wrapped position by engaging and pulling guide segment 40 so that at least a proximal region of the fixation segment follows the guide segment along a path through the tissue opening and around bone. In some embodiments, the fixation segment may have a length that is greater than the circumference of bone 62, so that all of the fixation segment can be spaced from bone (and external to the appendage) when the fixation segment is properly wrapped around the bone. In some embodiments, the fixation segment may have a length that is at least about twice the circumference, so that the fixation segment can be wrapped around the bone at least twice.

FIG. 8 shows tension, indicated by arrow 78, being applied to the wire with a tensioner 80, to tighten the wire around the bone. The tensioner may engage the fixation segment and/or the guide segment. The tensioner also may engage a portion of the appendage such as the bone or soft tissue, and/or may engage locking mechanism 32. The tensioner may tighten the wire by, for example, rotary movement of a portion (or all) of the tensioner, indicated at 81. In some embodiments, the tensioner may apply tension to the fixation segment by engagement of the guide segment. Accordingly, any junction region disposed between these segments should be strong enough to transmit tension to the fixation segment without breaking when the wire is tensioned through the guide segment.

FIG. 9 shows the wire being cut with wire cutters 82, after the wire has been secured in position around the bone. Accordingly, clamp device 32 may be actuated prior to cutting the wire (or after cutting the wire). In some embodiments, the wire may be twisted, tied, or manipulated otherwise to secure the wire around the bone. The step of cutting the wire may separate at least a substantial portion of the guide segment from the fixation segment. In some embodiments, the step of cutting may be performed within the fixation segment, so that all of the guide segment is removed from the remainder of the wire. In some embodiments, the wire also or alternatively may be cut in the fixation segment, such as to remove a proximal end of the wire, for example, stop element 36 and associated regions of the wire.

FIG. 10 shows the fractured bone with the wire system fixing the bone. Tissue opening 66 may be closed, such as with sutures, staples, tape, and/or the like.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

I claim:

1. A wire system for fixing a bone, comprising a unitary wire configured to wrap at least substantially around the bone and including a first segment and a second segment arrayed longitudinally along the wire, the first segment and the second segment each being formed of a material, the material of the first segment being more intrinsically flexible than the material of the second segment.

2. The wire system of claim 1, wherein the wire has a pair of opposing ends, and wherein the first segment extends at least substantially to one of the pair of ends and the second segment extends at least substantially to the other of the pair of ends.

3. The wire system of claim 1, wherein the material has at least substantially the same composition in each of the first and second segments.

4. The wire system of claim 1, wherein one of the segments has been at least one of selectively heated and selectively cooled relative to the other segment.

5. The wire system of claim 4, wherein the first segment has been selectively annealed.

6. The wire system of claim 1, wherein the first segment and the second segment have at least substantially the same diameter.

7. The wire system of claim 1, further comprising a stop element attached to the first segment and spaced from the second segment, the stop element having a characteristic width greater than the diameter of the first segment.

8. The wire system of claim 7, wherein the stop element is at least one of a bead and a loop.

9. The wire system of claim 1, wherein each of the first and second segments is formed at least substantially of stainless steel.

10. The wire system of claim 1, further comprising a locking mechanism configured to engage the wire so that the wire is secured around the bone.

11. A wire system for fixing a bone, comprising a wire configured to wrap at least substantially around the bone and including a first segment and a second segment arrayed longitudinally along the wire and having at least substantially the same diameter, the first segment being more flexible than the second segment.

12. A method of fixing a bone, comprising:
selecting a bone to be fixed;
selecting a wire including a relatively stiffer segment and a relatively more flexible segment arrayed longitudinally along the wire;
advancing the stiffer segment at least substantially around the bone so that the more flexible segment follows the stiffer segment until the flexible segment is wrapped at least substantially around the bone; and
securing the more flexible segment on the bone.

13. The method of claim 12, the bone having a discontinuity and a circumference measured adjacent the discontinuity, the stiffer segment and the more flexible segment each having a length, wherein the step of selecting a wire selects a wire in which the length of each of the stiffer segment and the more flexible segment is greater than the circumference.

14. The method of claim 12, soft tissue adjacent the bone including an opening to the bone, the stiffer segment having a leading end region spaced from the more flexible segment, wherein the step of advancing includes a step of feeding the stiffer segment inward through the opening until the leading end region of the stiffer segment has traveled at least substantially around the bone and passed outward through the opening.

15. The method of claim 12, wherein the step of advancing includes a step of selectively engaging the stiffer segment and a step of moving the stiffer segment after the step of selectively engaging.

16. The method of claim 15, the stiffer segment including a leading end region, wherein the step of moving includes a step of pushing the leading end region at least substantially around the bone and a step of pulling the leading end region away from the bone after the step of pushing.

17. The method of claim 12, further comprising a step of cutting the wire within the more flexible segment after the step of securing.

18. A method of fixing a bone, comprising:
selecting a bone to be fixed;
selecting a wire including a relatively stiffer segment and a relatively more flexible segment arrayed longitudinally along the wire;
positioning the more flexible segment at least substantially around the bone through selective engagement and manipulation of the stiffer segment; and
securing the more flexible segment on the bone.

19. A method of forming a device for fixing a bone, comprising:
selecting a wire configured to be wrapped around a bone; and
treating a longitudinal portion of the wire selectively to create a relatively stiffer segment and a relatively more flexible segment of the wire having at least substantially the same diameter.

20. The method of claim 19, the longitudinal portion corresponding to the more flexible segment, wherein the step of treating includes a step of selectively annealing the longitudinal portion.

21. The method of claim 19, further comprising a step of attaching a stop structure to the more flexible segment and spaced from the stiffer segment.

22. The method of claim 19, further comprising a step of coupling the more flexible segment to a locking mechanism that is actuable to secure the more flexible segment to the locking mechanism.
